(19) United States
(12) Patent Application Publication
(10) Pub. No.: US 2026/0233212 A1
(43) Pub. Date: Aug. 13, 2026

(54) REDUCED METHANE BYPRODUCT FORMATION IN METHANOL SYNTHESIS USING INDIUM-BASED CATALYSTS

(71) Applicants: Saudi Arabian Oil Company, Dhahran (SA); King Abdullah University of Science and Technology, Thuwal (SA)

(72) Inventors: Jorge Gascon Sabate, Thuwal (SA); Jean Marcel Ribeiro Gallo, Thuwal (SA); Wei Wang, Dengzhou City (CN); Mohnnad H. Alabsi, Thuwal (SA)

(73) Assignees: Saudi Arabian Oil Company, Dhahran (SA); King Abdullah University of Science and Technology, Thuwal (SA)

(21) Appl. No.: 19/048,096

(22) Filed: Feb. 7, 2025

(51) Int. Cl. B01J 37/03; B01J 23/825; B01J 37/08; B01J 37/18; C07C 29/154

(52) U.S. Cl. CPC B01J 37/036; B01J 23/825; B01J 37/088; B01J 37/18; C07C 29/154

(57) ABSTRACT

A method for preparing catalyst particles comprising preparing a precursor solution comprising a solvent, an indium-based composition, and at least one of a cobalt-based composition and a copper-based composition, contacting the precursor solution with an acidic solution to form a colloidal suspension, heating the colloidal suspension to form a gel, drying the gel to yield a solid material, and calcining the solid material to form catalyst particles, wherein the catalyst particles comprises a composite having at least one of indium oxide and indium hydroxide, and at least one of copper oxide, copper hydroxide, cobalt oxide, cobalt hydroxide, or combinations thereof.

### TECHNICAL FIELD

Embodiments disclosed herein generally relate to a catalyst and methods for preparing the catalyst.

### BACKGROUND

Methanol is a chemical that is currently of high importance and demand. Methanol synthesis traditionally relies on fossil fuel-derived syngas, wherein the syngas is most often sourced from natural gas reforming or coal gasification, which contribute to approximately 10% of carbon dioxide emissions within the chemical and petrochemical industries. Traditionally, methanol is synthesized using a conventional catalyst, for example a benchmark Cu—ZnO—Al₂O₃ catalyst, and CO₂, CO, and H₂ mixtures. To take action to reduce carbon dioxide emissions, there is an interest in producing methanol through hydrogenation of waste streams containing carbon dioxide, repurposing carbon dioxide emissions as a feed stream for methanol synthesis. Catalysts prepared by conventional methods may deactivate in the presence of common contaminants such as oxygen, aromatics, methane, and carbon monoxide. Further, catalysts prepared by conventional methods may result in relatively high methane selectivity when used for methanol synthesis. Methane formation can significantly affect the cost of methanol production, and is undesired for at least this reason. Low methane selectivity during hydrogenation of carbon dioxide to produce methanol is desirable for at least this reason. Further, common contaminants may be present in a waste streams comprising carbon dioxide, in which the contaminants may comprise oxygen, methane, carbon monoxide, and aromatics, which may include toluene, and combinations thereof. Catalysts prepared by conventional methods may be unstable in the presence of contaminants, and may not revert to initial catalytic activity or performance after contacting contaminants.

### SUMMARY

Methanol catalysts comprising indium-cobalt have been produced via wet impregnation, precipitation or co-precipitation, ball milling, and metal-organic framework (MOF)-mediated synthesis. These indium-cobalt catalysts convert carbon dioxide via hydrogenation at temperatures as high as 300° C.; however, contaminants, such as oxygen, toluene, may negatively affect the methanol selectivity and conversely increase methane yield. As a result, there is a need for a catalyst, and a method for producing a catalyst, that is stable in the presence of contaminants and suitable for hydrogenation of carbon dioxide to produce methanol with increased methanol selectivity and reduced methane selectivity.

The method for preparing catalyst particles embodiments of the present disclosure surprisingly meet this need for increased methanol selectivity while reducing the impact of contaminants.

According to one or more embodiments, a method for preparing catalyst particles may comprise preparing a precursor solution comprising a solvent, an indium-based composition, and at least one of a cobalt-based composition and a copper-based composition, contacting the precursor solution with an acidic solution to form a colloidal suspension, heating the colloidal suspension to form a gel, drying the gel to yield a solid material, and calcining the solid to form the catalyst particles, wherein the catalyst particles comprises a composite having at least one of an indium oxide, an indium hydroxide, or combinations thereof, and at least one of a copper oxide, a copper hydroxide, a cobalt oxide, a cobalt hydroxide, or combinations thereof.

This summary is provided to introduce a selection of concepts that are further described in the detailed description. This summary is not intended to identify key or essential features of the claimed subject matter, nor is it intended to be used as an aid in limiting the scope of the claimed subject matter.

Additional features and advantages of the embodiments described herein will be set forth in the detailed description that follows, and in part will be readily apparent to those skilled in the art from that description or recognized by practicing the embodiments described herein, including the detailed description that follows, and the claims.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 Percentual variation in methanol yield and (mol methanol)/(mol CH₄) ratio of Example Catalysts as compared to Comparative A.

FIG. 2 Percentual variation in methanol yield and (mol methanol)/(mol CH₄) ratio between InCo Example Catalysts.

FIG. 3 Percentual variation in methanol yield and (mol methanol)/(mol CH₄) ratio between InCoCu Example Catalysts.

FIG. 4 High performing Example Catalysts selectivity in comparison to Comparative A.

FIG. 5A Conversion and selectivity while co-feeding O₂ with Reduced Example Catalyst InCo-10/1.

FIG. 5B Conversion and selectivity while co-feeding O₂ with Reduced Example Catalyst InCo-5/1.

FIG. 5C Conversion and selectivity while co-feeding O₂ with Reduced Example Catalyst InCoCu-10/1/1.

FIG. 6A Conversion and selectivity while co-feeding CH₄ with Reduced Example Catalyst InCo-10/1.

FIG. 6B Conversion and selectivity while co-feeding CH₄ with Reduced Example Catalyst InCoCu-10/1/1.

FIG. 7A Conversion and selectivity while co-feeding CO with Reduced Example Catalyst InCo-5/1.

FIG. 7B Conversion and selectivity while co-feeding CO with Reduced Example Catalyst InCoCu-10/1/1.

FIG. 8A Conversion and selectivity while co-feeding toluene with Reduced Example Catalyst InCo-5/1.

FIG. 8B Conversion and selectivity while co-feeding toluene with Reduced Example Catalyst InCoCu-10/1/1.

### DETAILED DESCRIPTION

Presently described, according to one or more embodiments, are catalysts for methanol synthesis, methods for producing the catalysts, methods for activating the catalysts to produce activated catalysts, and methods for using the catalysts. Catalysts described in this disclosure may be made by sol-gel synthesis. Catalysts described in this disclosure may be unreduced unless specified otherwise.

The terms recited below have been defined as described below. All other terms and phrases in this disclosure shall be construed according to their ordinary meaning as understood by one of skill in the art.

As used in this disclosure, a “catalyst” refers to any substance that increases the rate of a specific chemical reaction. Catalysts described in this disclosure may be utilized to promote various reactions, such as, but not limited to, producing methanol by carbon dioxide hydrogenation.

As used in this disclosure, an “effluent” generally refers to a stream that exits a system component such as a separation unit, a reactor, or reaction zone, following a separation or particular reaction, and generally has a different composition (at least proportionally) than the stream that entered the separation unit, reactor, or reaction zone.

As used in this disclosure, “product effluent” generally refers to a stream that exits a system component such as a reactor or reaction zone, following a particular reaction, and generally has a different composition (at least proportionally) than the stream that entered the reactor or reaction zone.

As used in this disclosure, the “reaction zone” may be defined as a zone in the reactor containing the catalysts where the reaction is initiated.

As used in this disclosure, “methanol synthesis” generally refers to a chemical process that comprises producing methanol by reacting synthesis gas (for example, synthesis gas may comprise H₂, CO, CO₂, and combinations thereof) at a specific temperature and pressure in the presence of a catalyst. For example, methanol synthesis may refer to a chemical process that is characterized by the addition of hydrogen to carbon monoxide or of carbon dioxide compound structures. An example of methanol synthesis may comprise formulas: CO₂+3H₂→CH₃OH+H₂O and/or CO+2H₂→CH₃OH. An example of methanol synthesis may comprise hydrogenation of carbon dioxide to methanol.

As used in this disclosure, “sol” generally refers to a colloidal solution formed by polymerization of a sol-gel precursor.

As used in this disclosure, “sol-gel” or “gel” generally refers to a product formed by further polymerization of a sol, in which networked polymeric chains are formed.

As used in this disclosure, “sol-gel method” generally refers to a wet chemical process used to synthesize solid materials from a sol. For example, a solution of soluble metal salts may form a colloidal solution, or a sol, which link together to form a coherent network, or gel. This method may allow for control over structure and composition of final material through controlled chemical processes. Examples of controlled chemical processes may include hydrolysis, polymerization, gelation, condensation, drying, and densification. Embodiments of a method for preparing catalyst particles may comprise a sol-gel method.

As used in this disclosure, “water-soluble salt” generally describes an ionic compound that readily dissolves in water. Example of water-soluble salt may include nitrates, metal citrates, lactates, formates, acetates, sulfates, carbonates, chlorides, citrate, bromides, EDTA salts, oxalates, benzoates, stearates, and tartrates.

As used in this disclosure, “gas hourly space velocity” or “GHSV” is defined as the rate at which gases flow through a reactor relative to the weight of the catalyst. For example, GHSV may be calculated by dividing the volumetric gas flow rate per hour (L h⁻¹) by the catalyst weight in grams.

As used in this disclosure, “static condition” or “static conditions” generally describe a state in which the material is not agitated, and material is at rest and without external force exerted to the material. For example, no stir bars, impellers, or the like may be applied.

In embodiments of a method for preparing catalyst particles, the method may comprise preparing a precursor solution comprising a solvent, an indium-based composition, and at least one of a cobalt-based composition and a copper-based composition, contacting the precursor solution with an acidic solution to form a sol, heating the sol to form a gel, drying the gel to yield a solid material, and calcining the solid material to form catalyst particles, wherein the catalyst particles comprises a composite having at least one of an indium oxide, an indium hydroxide, or combinations thereof, and at least one of a copper oxide, a copper hydroxide, a cobalt oxide, a cobalt hydroxide, or combinations thereof. Without being bound by any particular theory, preparing catalyst particles for methanol synthesis by this method improves the selectivity of unwanted methane that comparable catalyst compositions made by other means may produce, from 4 to 8 percent, to a selectivity of methane below 1 percent.

In embodiments, the precursor solution may comprise an indium-based composition, and at least one cobalt-based composition. In one embodiment, the precursor solution comprises an indium-based composition and a copper-based composition. In another embodiment, the precursor solution comprises an indium-based composition, a copper-based composition, and a cobalt-based composition.

At least one or all three of the indium-based compositions, the copper-based composition, and the cobalt-based composition may comprise water-soluble salts. For example, nitrates. Thus, the precursor solution may comprise indium nitrate, copper nitrate, and cobalt nitrate. In addition to water-soluble salts, it is contemplated that the precursor solution may comprise other salts, such as metal citrates, lactates, formates, acetates, sulfates, carbonates, chlorides, citrate, bromides, EDTA salts, oxalates, benzoates, stearates, and tartrates.

As stated above, the precursor solution is contacted with an acidic solution to form a sol. In one embodiment, acidic solution may comprise an organic acid. In another embodiment, the organic acid may comprise a citric acid. In one embodiment, the pH of the organic acid is from 2 to 6. For instance, in further embodiments, the pH may be from 2 to 6, from 2 to 5.7, from 2 to 5.3, from 2 to 5, from 2 to 4.7, from 2 to 4.3, from 2 to 4, from 2 to 3.7, from 2 to 3.3, from 2 to 3, from 2 to 2.7, from 2 to 2.3, from 2.3 to 6, from 2.3 to 5.7, from 2.3 to 5.3, from 2.3 to 5, from 2.3 to 4.7, from 2.3 to 4.3, from 2.3 to 4, from 2.3 to 3.7, from 2.3 to 3.3, from 2.3 to 3, from 2.3 to 2.7, from 2.7 to 6, from 2.7 to 5.7, from 2.7 to 5.3, from 2.7 to 5, from 2.7 to 4.7, from 2.7 to 4.3, from 2.7 to 4, from 2.7 to 3.7, from 2.7 to 3.3, from 2.7 to 3, from 3 to 6, from 3 to 5.7, from 3 to 5.3, from 3 to 5, from 3 to 4.7, from 3 to 4.3, from 3 to 4, from 3 to 3.7, from 3 to 3.3, from 3.3 to 6, from 3.3 to 5.7, from 3.3 to 5.3, from 3.3 to 5, from 3.3 to 4.7, from 3.3 to 4.3, from 3.3 to 4, from 3.3 to 3.7, from 3.7 to 6, from 3.7 to 5.7, from 3.7 to 5.3, from 3.7 to 5, from 3.7 to 4.7, from 3.7 to 4.3, from 3.7 to 4, from 4 to 6, from 4 to 5.7, from 4 to 5.3, from 4 to 5, from 4 to 4.7, from 4 to 4.3, from 4.3 to 6, from 4.3 to 5.7, from 4.3 to 5.3, from 4.3 to 5, from 4.3 to 4.7, from 4.7 to 6, from 4.7 to 5.7, from 4.7 to 5.3, from 4.7 to 5, from 5 to 6, from 5 to 5.7, from 5 to 5.3, from 5.3 to 6, from 5.3 to 5.7, or from 5.7 to 6.

In embodiments, the precursor solution comprises one or more metal. In at least one embodiment, the precursor solution comprises from 10 mol. % to 98 mol. % indium, based on the total metal content. For example, the precursor solution may comprise from 10 mol. % to 98 mol. %, from 10 mol. % to 83 mol. %, from 10 mol. % to 69 mol. %, from 10 mol. % to 54 mol. %, from 10 mol. % to 39 mol. %, from 10 mol. % to 25 mol. %, from 12.5 mol. % to 95.2 mol. % from 25 mol. % to 98 mol. %, from 25 mol. % to 83 mol. %, from 25 mol. % to 69 mol. %, from 25 mol. % to 54 mol. %, from 25 mol. % to 39 mol. %, from 39 mol. % to 98 mol. %, from 39 mol. % to 83 mol. %, from 39 mol. % to 69 mol. %, from 39 mol. % to 54 mol. %, from 54 mol. % to 98 mol. %, from 54 mol. % to 83 mol. %, from 54 mol. % to 69 mol. %, from 69 mol. % to 98 mol. %, from 69 mol. % to 83 mol. %, or from 83 mol. % to 98 mol. % indium, based on the total metal content of the precursor solution.

In embodiments, the precursor solution comprises one or more metal. In at least one embodiment, the precursor solution comprises up to 90.0 mol. % cobalt, based on the total metal content of the precursor solution. For example, the precursor solution may comprise up to 85.7 mol. %, up to 85 mol. %, up to 80 mol. %, up to 75 mol. %, up to 70 mol. %, up to 65 mol. %, up to 60 mol. %, up to 55 mol. %, up to 50 mol. %, up to 45 mol. %, up to 40 mol. %, up to 35 mol. %, up to 30 mol. %, up to 25 mol. %, up to 20 mol. %, up to 15 mol. %, up to 10 mol. %, or up to 5 mol. % cobalt, based on the total metal content of the precursor solution.

In embodiments, the precursor solution comprises one or more metal. In at least one embodiment, the precursor solution comprises up to 40.0 mol. % copper, based on the total metal content of the precursor solution. For example, the precursor solution may comprise up to 35 mol. %, up to 30 mol. %, up to 25 mol. %, up to 20 mol. %, up to 15 mol. %, up to 10 mol. %, or up to 5 mol. % copper, based on the total metal content of the precursor solution.

As stated above, the method for preparing catalyst particles may comprise heating the sol to form a gel, in which the heating may take place at from 50° C. to 90° C. For instance, the temperature at which heating of the sol to form a gel may be from 50° C. to 90° C., from 50° C. to 85° C., from 50° C. to 80° C., from 50° C. to 75° C., from 50° C. to 70° C., from 50° C. to 65° C., from 50° C. to 60° C., from 50° C. to 55° C., from 55° C. to 90° C., from 55° C. to 85° C., from 55° C. to 80° C., from 55° C. to 75° C., from 55° C. to 70° C., from 55° C. to 65° C., from 55° C. to 60° C., from 60° C. to 90° C., from 60° C. to 85° C., from 60° C. to 80° C., from 60° C. to 75° C., from 60° C. to 70° C., from 60° C. to 65° C., from 65° C. to 90° C., from 65° C. to 85° C., from 65° C. to 80° C., from 65° C. to 75° C., from 65° C. to 70° C., from 70° C. to 90° C., from 70° C. to 85° C., from 70° C. to 80° C., from 70° C. to 75° C., from 75° C. to 90° C., from 75° C. to 85° C., from 75° C. to 80° C., from 80° C. to 90° C., from 80° C. to 85° C., or from 85° C. to 90° C.

As stated above, the method for preparing catalyst particles may comprise heating the sol to form a gel, in which the heating may take place for a duration of from 0.5 to 5 hours. For instance, the duration of heating of the sol to form a gel may be from 0.5 hr to 5 hr, from 0.5 hr to 4 hr, from 0.5 hr to 4 hr, from 0.5 hr to 3 hr, from 0.5 hr to 3 hr, from 0.5 hr to 2 hr, from 0.5 hr to 2 hr, from 0.5 hr to 1 hr, from 1.1 hr to 5 hr, from 1.1 hr to 4 hr, from 1.1 hr to 4 hr, from 1.1 hr to 3 hr, from 1.1 hr to 3 hr, from 1.1 hr to 2 hr, from 1.1 hr to 2 hr, from 1.6 hr to 5 hr, from 1.6 hr to 4 hr, from 1.6 hr to 4 hr, from 1.6 hr to 3 hr, from 1.6 hr to 3 hr, from 1.6 hr to 2 hr, from 2.2 hr to 5 hr, from 2.2 hr to 4 hr, from 2.2 hr to 4 hr, from 2.2 hr to 3 hr, from 2.2 hr to 3 hr, from 2.8 hr to 5 hr, from 2.8 hr to 4 hr, from 2.8 hr to 4 hr, from 2.8 hr to 3 hr, from 3.3 hr to 5 hr, from 3.3 hr to 4 hr, from 3.3 hr to 4 hr, from 3.9 hr to 5 hr, from 3.9 hr to 4 hr, or from 4.4 hr to 5 hr.

As stated above, the method for preparing catalyst particles may comprise drying the gel to yield a solid material, in which drying may take place at a temperature from 90° C.° C. to 150° C.° C. For instance, in embodiments the drying temperature may be from 90° C.° C. to 150° C.° C., from 90° C.° C. to 143° C.° C., from 90° C. to 135° C., from 90° C. to 128° C., from 90° C. to 120° C., from 90° C. to 113° C., from 90° C. to 105° C., from 90° C. to 98° C., from 98° C. to 150° C., from 98° C. to 143° C., from 98° C. to 135° C., from 98° C. to 128° C., from 98° C. to 120° C., from 98° C. to 113° C., from 98° C. to 105° C., from 105° C. to 150° C., from 105° C. to 143° C., from 105° C. to 135° C., from 105° C. to 128° C., from 105° C. to 120° C., from 105° C. to 113° C., from 113° C. to 150° C., from 113° C. to 143° C., from 113° C. to 135° C., from 113° C. to 128° C., from 113° C. to 120° C., from 120° C. to 150° C., from 120° C. to 143° C., from 120° C. to 135° C., from 120° C. to 128° C., from 128° C. to 150° C., from 128° C. to 143° C., from 128° C. to 135° C., from 135° C. to 150° C., from 135° C. to 143° C., or from 143° C. to 150° C.

The calcining of the solid material may occur at a temperature from 250° C. to 500° C. to form unreduced catalyst particles. For instance, in embodiments the calcining temperature may be from 250° C. to 500° C., from 250° C. to 469° C., from 250° C. to 438° C., from 250° C. to 406° C., from 250° C. to 375° C., from 250° C. to 344° C., from 250° C. to 313° C., from 250° C. to 281° C., from 281° C. to 500° C., from 281° C. to 469° C., from 281° C. to 438° C., from 281° C. to 406° C., from 281° C. to 375° C., from 281° C. to 344° C., from 281° C. to 313° C., from 313° C. to 500° C., from 313° C. to 469° C., from 313° C. to 438° C., from 313° C. to 406° C., from 313° C. to 375° C., from 313° C. to 344° C., from 344° C. to 500° C., from 344° C. to 469° C., from 344° C. to 438° C., from 344° C. to 406° C., from 344° C. to 375° C., from 375° C. to 500° C., from 375° C. to 469° C., from 375° C. to 438° C., from 375° C. to 406° C., from 406° C. to 500° C., from 406° C. to 469° C., from 406° C. to 438° C., from 438° C. to 500° C., from 438° C. to 469° C., or from 469° C. to 500° C.

In embodiments, the method for preparing catalyst particles may comprise a catalyst reduction step. The reduction step may comprise contacting unreduced catalyst particles to a reduction gas feed under an initial temperature and increasing to a final temperature to activate the catalyst particles to form reduced catalyst particles. In at least one embodiment, the unreduced catalyst were employed for methanol synthesis without the additional catalyst reduction step. In at least one embodiment, the unreduced catalyst were reduced by the additional catalyst reduction step, resulting in activated catalyst particles.

The reduction gas feed may comprise from 25 vol. % to 95 vol. % nitrogen. For instance, in embodiments the volume of nitrogen in the reduction gas feed may be from 25 vol. %. to 95 vol. %., from 25 vol. %. to 85 vol. %., from 25 vol. %. to 75 vol. %., from 25 vol. %. to 65 vol. %., from 25 vol. %. to 55 vol. %., from 25 vol. %. to 45 vol. %., from 25 vol. %. to 35 vol. %., from 35 vol. %. to 95 vol. %., from 35 vol. %. to 85 vol. %., from 35 vol. %. to 75 vol. %., from 35 vol. %. to 65 vol. %., from 35 vol. %. to 55 vol. %., from 35 vol. %. to 45 vol. %., from 45 vol. %. to 95 vol. %., from 45 vol. %. to 85 vol. %., from 45 vol. %. to 75 vol. %., from 45 vol. %. to 65 vol. %., from 45 vol. %. to 55 vol. %., from 55 vol. %. to 95 vol. %., from 55 vol. %. to 85 vol. %., from 55 vol. %. to 75 vol. %., from 55 vol. %. to 65 vol. %., from 65 vol. %. to 95 vol. %., from 65 vol. %. to 85 vol. %., from 65 vol. %. to 75 vol. %., from 75 vol. %. to 95 vol. %., from 75 vol. %. to 85 vol. %., or from 85 vol. %. to 95 vol. %.

Moreover, the reduction gas feed may comprise up to 100 vol. % hydrogen. For instance, in embodiments the volume of hydrogen in the reduction gas feed may be from up to 99 vol. %, up to 95 vol. %, up to 90 vol. %, up to 85 vol. %, up to 80 vol. %, up to 75 vol. %, up to 70 vol. %, up to 65 vol. %, up to 60 vol. %, up to 55 vol. %, up to 50 vol. %, up to 45 vol. %, up to 40 vol. %, up to 35 vol. %, up to 30 vol. %, up to 25 vol. %, up to 20 vol. %, up to 15 vol. %, up to 99 vol. %, up to 10 vol. %, or up to 5 vol. %.

Furthermore, the initial reduction temperature may be from 25° C. to 175° C. For instance, in embodiments the initial reduction temperature may be from 25° C. to 175° C., from 25° C. to 156° C., from 25° C. to 138° C., from 25° C. to 119° C., from 25° C. to 100° C., from 25° C. to 81° C., from 25° C. to 63° C., from 25° C. to 44° C., from 44° C. to 175° C., from 44° C. to 156° C., from 44° C. to 138° C., from 44° C. to 119° C., from 44° C. to 100° C., from 44° C. to 81° C., from 44° C. to 63° C., from 63° C. to 175° C., from 63° C. to 156° C., from 63° C. to 138° C., from 63° C. to 119° C., from 63° C. to 100° C., from 63° C. to 81° C., from 81° C. to 175° C., from 81° C. to 156° C., from 81° C. to 138° C., from 81° C. to 119° C., from 81° C. to 100° C., from 100° C. to 175° C., from 100° C. to 156° C., from 100° C. to 138° C., from 100° C. to 119° C., from 119° C. to 175° C., from 119° C. to 156° C., from 119° C. to 138° C., from 138° C. to 175° C., from 138° C. to 156° C., or from 156° C. to 175° C.

The final reduction temperature may be from 250° C. to 600° C. For instance, in embodiments the final reduction temperature may be from 250° C. to 600° C., from 250° C. to 556° C., from 250° C. to 513° C., from 250° C. to 469° C., from 250° C. to 425° C., from 250° C. to 381° C., from 250° C. to 338° C., from 250° C. to 294° C., from 294° C. to 600° C., from 294° C. to 556° C., from 294° C. to 513° C., from 294° C. to 469° C., from 294° C. to 425° C., from 294° C. to 381° C., from 294° C. to 338° C., from 338° C. to 600° C., from 338° C. to 556° C., from 338° C. to 513° C., from 338° C. to 469° C., from 338° C. to 425° C., from 338° C. to 381° C., from 381° C. to 600° C., from 381° C. to 556° C., from 381° C. to 513° C., from 381° C. to 469° C., from 381° C. to 425° C., from 425° C. to 600° C., from 425° C. to 556° C., from 425° C. to 513° C., from 425° C. to 469° C., from 469° C. to 600° C., from 469° C. to 556° C., from 469° C. to 513° C., from 513° C. to 600° C., from 513° C. to 556° C., or from 556° C. to 600° C.

The reduction step may comprise contacting the unreduced catalyst particles to a reduction gas feed under an initial temperature and increasing to a final temperature from between 0.5 to 10 hours to form reduced catalyst particles. For instance, in embodiments the reducing duration may be from 0.5 hours to 10 hours, from 1 hours to 9 hours, from 1 hours to 7 hours, from 1 hours to 6 hours, from 1 hours to 5 hours, from 1 hours to 3 hours, from 1 hours to 2 hours, from 2 hours to 10 hours, from 2 hours to 9 hours, from 2 hours to 7 hours, from 2 hours to 6 hours, from 2 hours to 5 hours, from 2 hours to 3 hours, from 3 hours to 10 hours, from 3 hours to 9 hours, from 3 hours to 7 hours, from 3 hours to 6 hours, from 3 hours to 5 hours, from 5 hours to 10 hours, from 5 hours to 9 hours, from 5 hours to 7 hours, from 5 hours to 6 hours, from 6 hours to 10 hours, from 6 hours to 9 hours, from 6 hours to 7 hours, from 7 hours to 10 hours, from 7 hours to 9 hours, or from 9 hours to 10 hours.

Moreover, the reduction gas feed may enter the reactor at a space velocity from 5 L_{(H2+N2)} g_{cat}⁻¹ h⁻¹ to 100 L_{(H2+N2)} g_{cathu -1} h⁻¹L_{(H2+N2)} g_{cat}⁻¹ h⁻¹L_{(H2+N2)} g_{cat}⁻¹ h⁻¹. For instance, the space velocity may be may be from 5 L_{(H2+N2)} g_{cathu -1} h⁻¹ to 100 L_{(H2+N2)} g_{cat}⁻¹ h⁻¹, from 5 L_{(H2+N2)} g_{cat}⁻¹ h⁻¹ to 84 L_{(H2+N2)} g_{cat}⁻¹ h⁻¹, from 5 L_{(H2+N2)} g_{cathu -1} h⁻¹ to 68 L_{(H2+N2)} g_{cat}⁻¹ h⁻¹, from 5 L_{(H2+N2)} g_{cat}⁻¹ h⁻¹ to 53 L_{(H2+N2)} g_{cathu -1} h⁻¹, from 5 L_{(H2+N2)} g_{cathu -1} h⁻¹ to 37 L_{(H2+N2)} g_{cat}⁻¹ h⁻¹, from 5 L_{(H2+N2)} g_{cathu -1} h⁻¹ to 21 L_{(H2+N2)} g_{cathu -1} h⁻¹, from 21 L_{(H2+N2)} g_{cathu -1} h⁻¹ to 100 L_{(H2+N2)} g_{cat}⁻¹ h⁻¹, from 21 L_{(H2+N2)} g_{cathu -1} h⁻¹ to 84 L_{(H2+N2)} g_{cat}⁻¹ h⁻¹, from 21 L_{(H2+N2)} g_{cat}⁻¹ h⁻¹ to 68 L_{(H2+N2)} g_{cat}⁻¹ h⁻¹, from 21 L_{(H2+N2)} g_{cat}⁻¹ h⁻¹ to 53 L_{(H2+N2)} g_{cathu -1} h⁻¹, from 21 L_{(H2+N2)} g_{cathu -1} h⁻¹ to 37 L_{(H2+N2)} g_{cat}⁻¹ h⁻¹, from 37 L_{(H2+N2)} g_{cat}⁻¹ h⁻¹ to 100 L_{(H2+N2)} g_{cathu -1} h⁻¹, from 37 L_{(H2+N2)} g_{cat}⁻¹ h⁻¹ to 84 L_{(H2+N2)} g_{cat}⁻¹ h⁻¹, from 37 L_{(H2+N2)} g_{cat}⁻¹ h⁻¹ to 68 L_{(H2+N2)} g_{cat}⁻¹ h⁻¹, from 37 L_{(H2+N2)} g_{cathu -1} h⁻¹ to 53 L_{(H2+N2)} g_{cat}⁻¹ h⁻¹, from 53 L_{(H2+N2)} g_{cathu -1} h⁻¹ to 100 L_{(H2+N2)} g_{cat}⁻¹ h⁻¹, from 53 L_{(H2+N2)} g_{cat}⁻¹ h⁻¹ to 84 L_{(H2+N2)} g_{cathu -1} h⁻¹, from 53 L_{(H2+N2)} g_{cat}⁻¹ h⁻¹ to 68 L_{(H2+N2)} g_{cat}⁻¹ h⁻¹, from 68 L_{(H2+N2)} g_{cathu -1} h⁻¹ to 100 L_{(H2+N2)} g_{cat}⁻¹ h⁻¹, from 68 L_{(H2+N2)} g_{cat}⁻¹ h⁻¹ to 84 L_{(H2+N2)} g_{cat}⁻¹ h⁻¹, or from 84 L_{(H2+N2)} g_{cathu -1} h⁻¹ to 100 L_{(H2+N2)} g_{cathu -1} h⁻¹.

In embodiments of a process for producing methanol by hydrogenation with the catalyst particles formed by the method for preparing catalyst particles, the process may comprise contacting a mixed gas feed to unreduced catalyst particles at a reaction temperature, the mixed gas feed comprising carbon dioxide and hydrogen, to form a product stream comprising methanol.

In embodiments of a process for producing methanol by hydrogenation with the catalyst particles formed by the method for preparing catalyst particles, the process may comprise contacting a mixed gas feed to reduced catalyst particles at a reaction temperature, the mixed gas feed comprising carbon dioxide and hydrogen, to form a product stream comprising methanol.

A process for producing methanol by hydrogenation with the catalyst particles formed by the method for preparing catalyst particles, the process comprising contacting a mixed gas feed to catalyst particles at a reaction temperature, the mixed gas feed comprising carbon dioxide and hydrogen, to form a product stream comprising methanol and methane, wherein the selectivity of methane is less than 3.5%. For instance, the selectivity of methane may be less than or equal to 3%, 2.5%, 2%, 1.5%, 1%, or 0.5%.

A process for producing methanol by hydrogenation with the catalyst particles formed by the method for preparing catalyst particles, the process comprising contacting a mixed gas feed to catalyst particles at a reaction temperature, the mixed gas feed comprising carbon dioxide and hydrogen, to form a product stream comprising methanol and methane, wherein the molar ratio of methanol to methane is from 2:1 to 450:1. For instance, the molar ratio of methanol to methane may be from 2:1 to 450:1, from 2:1 to 375:1, from 2:1 to 301:1, from 2:1 to 226:1, from 2:1 to 151:1, from 2:1 to 77:1, from 77:1 to 450:1, from 77:1 to 375:1, from 77:1 to 301:1, from 77:1 to 226:1, from 77:1 to 151:1, from 151:1 to 450:1, from 151:1 to 375:1, from 151:1 to 301:1, from 151:1 to 226:1, from 226:1 to 450:1, from 226:1 to 375:1, from 226:1 to 301:1, from 301:1 to 450:1, from 301:1 to 375:1, or from 375:1 to 450:1.

A process for producing methanol by hydrogenation with the catalyst particles formed by the method for preparing catalyst particles, the process comprising contaminants, wherein the contaminants are selected from the group oxygen, methane, carbon monoxide, toluene, or combinations thereof.

A process for producing methanol by hydrogenation with the catalyst particles formed by the method for preparing catalyst particles, the process comprising contacting a mixed gas feed to catalyst particles at a reaction temperature, the mixed gas feed comprising carbon dioxide and hydrogen, to form a product stream comprising methanol and methane, the process further comprising contaminants, wherein the contaminants are selected from the group oxygen, methane, carbon monoxide, toluene, or combinations thereof, wherein the selectivity of methane is less than 3.5%. For instance, the selectivity of methane may be less than or equal to 3%, 2.5%, 2%, 1.5%, 1%, or 0.5%.

### EXAMPLES

The following Examples are offered by way of demonstration and are presented in a manner such that one skilled in the art should recognize are not meant to be limiting to the present disclosure as a whole or to the appended claims.

### Example Catalyst Synthesis

Catalysts comprising indium and one or more of cobalt and copper herein are synthesized by a sol-gel method. A method for preparing catalyst particles comprise this synthesis method. The catalysts were named according to the metals in their composition, i.e., a catalyst comprising indium and cobalt metals without copper may be referred to as “InCo” and a catalyst comprising indium, cobalt, and copper may be referred to as “InCoCu”, respectively. The method for preparing catalyst particles comprises contacting a precursor material, which comprises a first metal soluble salt and a second metal soluble salt, or a first metal soluble salt, a second metal soluble salt, and a third metal soluble salt, into water in a first vessel to form a precursor solution, contacting an acidifier to water in a second vessel to form a subsequent solution, contacting the precursor solution to the subsequent solution to form a synthesis solution, or “sol.” The sol was heated to form a gel, the gel was dried to form a solid, and the solid was calcined to form catalyst particles.

Two category Example Catalyst compositions are synthesized by the sol-gel method: Example Catalysts, which contain indium, cobalt, and copper (“InCoCu Example Catalysts”) and Example Catalysts, which contain indium and cobalt (“InCo Example Catalysts”). InCoCu Example Catalysts and InCo Example Catalysts are named for being comprised of their respective metal oxides and/or metal hydroxides, and are not, for example, comprised of metallic alloys. Catalysts are unreduced unless specified otherwise. For example, an unreduced indium-cobalt catalyst may be referred to as “InCo Example catalyst,” while its reduced counterpart may be referred to as “Reduced InCo Example Catalyst” or “Red-InCo.”

### Indium, Cobalt, and Copper-based Catalysts: InCoCu Example Catalyst Synthesis

Specifically, InCoCu Example Catalysts (indium, cobalt, and copper-based catalysts of the present example) are synthesized by the sol-gel method, wherein Table 1 summarizes the mass quantities of Indium Nitrate [In(NO₃)₃], cobalt nitrate hexahydrate [Co(NO₃)₂·6H₂O], and copper nitrate trihydrate [Cu(NO₃)₂·3H₂O] for each individual InCoCu Example Catalyst. For each catalyst, the specified amount of indium nitrate [In(NO₃) 3], cobalt nitrate hexahydrate [Co(NO₃)₂·6H₂O], and copper nitrate trihydrate [Cu(NO₃)₂·3H₂O] were dissolved into 6 mL of water to form an initial solution. A separate 500 mL vessel was charged with 8 mL of water, to which 7.7 g of citric acid was dissolved to form a subsequent solution. While agitating the subsequent solution, the initial solution was added gradually to form a InCoCu synthesis solution. The InCoCu synthesis solution was heated to 80° C. under agitation for 2 hours to form a gel. The gel was then dried at 120° C. under static conditions to form a solid. The solid was ground to form a powder. The powder was then calcined for 3 hours at 350° C. to form an unreduced InCoCu catalyst. This synthesis is repeated for each unreduced InCoCu catalyst prepared in Table 1, and the resulting molar ratios of indium, cobalt, and copper are summarized here as well. Each InCoCu Example Catalyst is identified by its composition, followed by its molar ratio of In, Co, and Cu. For example, an InCoCu Example Catalyst made from 3.008 g In(NO₃)₃, 1.455 g Co(NO₃)₂·6H₂O, and 1.208 g Cu(NO₃)₂·3H₂O may be designated “InCoCu-2/1/1,” which represents this specific InCoCu Example Catalyst with 2 moles indium for one mole of cobalt and one mole of copper (Table 1).

### Indium and Cobalt Catalysts: InCo Example Catalyst Synthesis

Specifically, InCo Example Catalysts (indium and cobalt-based catalyst of the present example) are synthesized by the sol-gel method, wherein Table 1 summarizes the mass quantities of Indium Nitrate [In(NO₃)₃] and cobalt nitrate hexahydrate [Co(NO₃)₂·6H₂O] for each individual InCo Example Catalyst. For each catalyst, the specified amount of indium nitrate [In(NO₃)₃] and cobalt nitrate hexahydrate [Co(NO₃)₂·6H₂O] were dissolved into 6 mL of water to form an initial, precursor solution. A separate 500 mL vessel was charged with 8 mL of water, to which 7.7 g of citric acid was dissolved to form a subsequent solution. While agitating the subsequent solution, the precursor solution was added gradually to form an InCo synthesis solution, an InCo sol. The InCo sol was heated to 80° C. under agitation for 2 hours to form a gel. The gel was dried at 120° C. under static conditions to form a solid. The solid was ground to form a powder. The powder was then calcined for 3 hours at 350° C. to form an unreduced InCo catalyst. This synthesis was repeated for each unreduced InCo catalyst prepared in Table 1, and the resulting molar ratios of indium and cobalt are summarized here as well. Each InCo Example Catalyst is identified by its composition, followed by its molar ratio of In and Co. For example, an InCo Example Catalyst made from 4.011 g In(NO₃)₃, 1.940 g Co(NO₃)₂·6H₂O, and 0.0 g Cu(NO₃)₂·3H₂O may be designated “InCo-2/1,” which represents this specific InCo Example Catalyst with 2 moles indium for one mole of cobalt (Table 1).

TABLE 1
Mass amount of metal nitrates used in the synthesis
of the InCoCu and InCo Example Catalysts.

| | | | | | | | Mass of Metal Nitrate (g) | | |
|---|---|---|---|---|---|---|---|---|---|
| | Composition (mol. %) | | | Molar ratio | | | | [Co(NO₃)₂· | [Cu(NO₃)₂· |
| Catalyst Name | In | Co | Cu | In | Co | Cu | [In(NO₃)₃] | 6H₂O] | 3H₂O] |
| InCu = 3/1 | 75.0 | 0.0 | 25.0 | 3 | 0 | 1 | 4.51 | 0 | 1.21 |
| InCoCu = 1/6/1 | 12.5 | 75.0 | 12.5 | 1 | 6 | 1 | 0.75 | 4.37 | 0.60 |
| InCoCu = 1/2/1 | 25.0 | 50.0 | 25.0 | 1 | 2 | 1 | 1.50 | 2.91 | 1.21 |
| InCoCu = 2/1/1 | 50.0 | 25.0 | 25.0 | 2 | 1 | 1 | 3.01 | 1.46 | 1.21 |
| InCoCu = 2/1/2 | 40.0 | 20.0 | 40.0 | 2 | 1 | 2 | 2.41 | 1.16 | 1.93 |
| InCoCu = 3.5/1/1 | 63.6 | 18.2 | 18.2 | 3.5 | 1 | 1 | 2.24 | 0.90 | 1.49 |
| InCoCu = 5/2/1 | 62.5 | 25.0 | 12.5 | 5 | 2 | 1 | 3.76 | 1.46 | 0.6 |
| InCoCu = 5/1/2 | 62.5 | 12.5 | 25.0 | 5 | 1 | 2 | 3.76 | 0.73 | 1.21 |
| InCoCu = 6/1/1 | 75.0 | 12.5 | 12.5 | 6 | 1 | 1 | 4.51 | 0.73 | 0.60 |
| InCoCu = 10/1/1 | 83.3 | 8.3 | 8.3 | 10 | 1 | 1 | 5.01 | 0.49 | 0.40 |
| InCoCu = 20/1/1 | 90.9 | 4.5 | 4.5 | 20 | 1 | 1 | 5.47 | 0.27 | 0.22 |
| InCoCu = 40/1/1 | 95.2 | 2.4 | 2.4 | 40 | 1 | 1 | 5.73 | 0.14 | 0.12 |
| InCo = 1/6 | 14.3 | 85.7 | 0.0 | 1 | 6 | 0 | 0.86 | 4.99 | 0 |
| InCo = 2/1 | 66.7 | 33.3 | 0.0 | 2 | 1 | 0 | 4.01 | 1.94 | 0 |
| InCo = 3/1 | 75.0 | 25.0 | 0.0 | 3 | 1 | 0 | 4.51 | 1.46 | 0 |
| InCo = 5/1 | 83.3 | 16.7 | 0.0 | 5 | 1 | 0 | 5.01 | 0.97 | 0 |
| InCo = 6/1 | 85.7 | 14.3 | 0.0 | 6 | 1 | 0 | 5.16 | 0.83 | 0 |
| InCo = 10/1 | 90.9 | 9.1 | 0.0 | 10 | 1 | 0 | 5.47 | 0.53 | 0 |
| InCo = 20/1 | 95.2 | 4.8 | 0.0 | 20 | 1 | 0 | 5.73 | 0.28 | 0 |

### Comparative Catalyst A Synthesis

Comparative Catalyst A is an InCo catalyst that was not formed by Sol-Gel synthesis. Specifically, Comparative Catalyst A was prepared by reverse co-precipitation, wherein the steps to prepare a catalyst by reverse co-precipitation comprises contacting a metal precursor solution with an alkaline aqueous solution to precipitate the catalyst. A detailed description of the catalyst preparation steps may be reviewed in U.S. Pat. No. 11,278,872 B2.

### Example Catalyst Performance

### Experiment Setup

Catalytic experiments were carried out in quartz reactors using a Flowrence® with sixteen parallel reactors system from Avantium. The quartz reactors, each reactor 20 cm in length with an internal diameter of 2 μm, were each filled with a 9.5 cm layer of coarse SiC (particle size 40, 300 μm) to establish an isothermal zone for the catalytic bed. After filling each reactor with coarse SiC, each reactor to be used for catalyst performance testing was charged with 50 mg of catalyst particles (particle size from 150 μm to 250 μm). Two quartz reactors were only packed with coarse SiC to perform blank tests as a procedural control.

The Flowrence® parallel reactor system from Avantium distributed mixed feed gas flow across 16 channels to sustain a relative flow rate standard deviation of 2%.

Prior to performing experiments, reactors were purged with N₂ for 1 hour at 100° C.

Unreduced catalyst performance was tested and measured to compare against reduced catalyst performance under identical reactor conditions. Only the treatment of the catalysts prior to performance tests differed, in which reactor conditions are adjusted to attain the desired catalyst reduction. To evaluate the performance of the unreduced catalysts, the reactors were first heated to the reaction temperature under a flow of N₂. Subsequently, the N₂ flow was replaced with the feed mixture, and the reactors were pressurized to the reaction pressure. To evaluate the performance of the reduced catalysts, the reactors were first heated to the reduction temperature under a flow of N₂. Then, the N₂ flow was replaced by a mixture of H₂ and inert gas for the required reduction time. Subsequently, the reactors were fed with the feed mixture, and the reactors were pressurized to the reaction pressure.

Reaction conditions are specific to the experiment conducted. Reaction conditions include at least a mixed gas feed composition, mixed gas feed space velocity, reaction temperature, and pressure. Example Catalysts were used in the reactor system without being reduced in order to produce performance results of unreduced Example Catalysts in Reactor Condition Example 1. Example catalysts were used in the reactor system and reduced in order to produce performance results of reduced Example Catalysts in Reactor Condition Example 2. Example Catalyst performances were measured by methanol synthesis reaction, unreacted reactants, and typical products, in which the reactants and products were analyzed with an Agilent 7890B gas chromatograph equipped with two loops. One loop was connected to a Colum 5 Flaysep Q 6 Ft G3591-80013 with TCD. One loop was connected to a Gaspro 30 MM OD column followed by FID.

### Methanol Synthesis Reaction Conditions

InCo Example Catalysts and InCoCu Example Catalysts were tested for performance by applying the catalysts to a methanol synthesis reaction, wherein the conditions of each methanol synthesis reaction performed comprises a mixed gas feed, wherein a mixed gas feed comprised 17.2 vol. % CO₂, 69.0 vol. % H₂, and 13.8 vol. % He, and the mixed gas feed was fed to the reactor at a space velocity of 15 L_{(CO2+H2)} g_{cat}⁻¹ h⁻¹. A membrane-based pressure controller operated with N₂ was used to pressurize the reactor to 50 bar.

### Example 1: Unreduced Catalyst Performance

### Example 1 Reactor Condition

InCo Example Catalysts and InCoCu Example Catalysts were tested as unreduced catalysts in Example 1 under methanol synthesis reaction conditions hereinabove. Specifically, a mixed gas feed of 17.2 vol. % CO₂, 69.0 vol. % H₂, and 13.8 vol. % He, and fed to the reactor at a space velocity of 15 L_{(CO2+H2)} g_{cat}⁻¹ h⁻¹. Reactor was heated to 300° C. A membrane-based pressure controller operated with N₂ was used to pressurize the reactor to 50 bar.

### Example 1 Results

Example 1 demonstrates that unreduced InCo Example Catalysts and unreduced InCoCu Example catalysts synthesized by the sol-gel method produce less methane compared to Example Catalyst A, which was not produced by the sol-gel method.

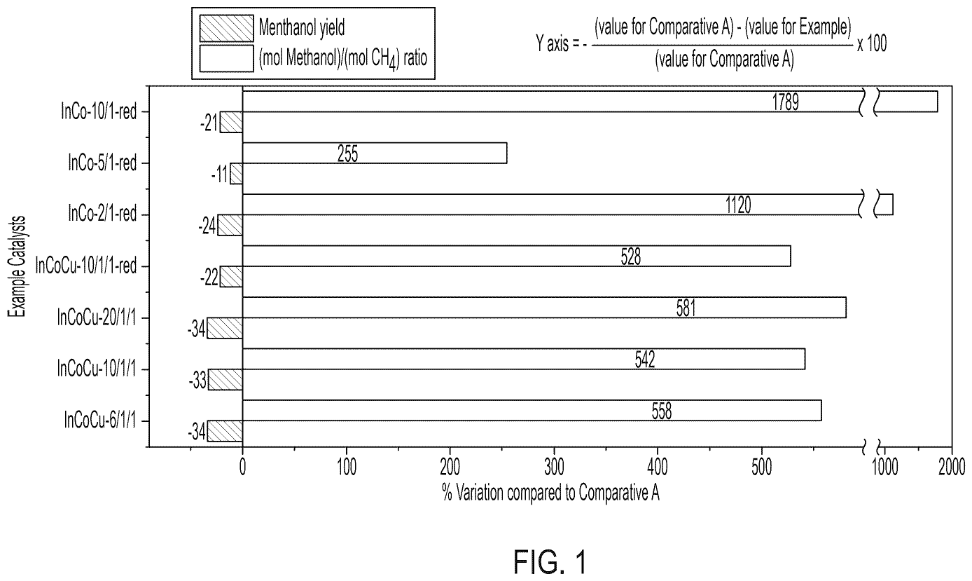

A summary of unreduced catalyst performance based on measured CO₂ conversion, CO selectivity, CH₄ selectivity, CH₄OH selectivity, methanol yield, CH₄ yield, and molar ratio of methanol to CH₄ is detailed in Table 2. Tests performed under Example 1 provide catalyst performance data under identical reactor conditions to compare catalyst performance by method of synthesizing and by composition. Specifically, FIG. 1 evaluates the data from Table 2 by diagramming the percent variation in methanol yield and molar ratio of methanol to CH₄ of sol-gel synthesized catalysts, as compared to Comparative Catalyst A, which was not synthesized by sol-gel.

TABLE 2
CO₂ hydrogenation to methanol using unreduced Example catalysts, InCo and
InCoCu, as compared to Comparative Catalyst A performance. Conversion and
selectivity obtained as the average and the error calculated as standard deviation.

| | | | | CO₂ | | | | | | (mol |
|---|---|---|---|---|---|---|---|---|---|---|
| | Molar ratio | | | Conv. | Selectivity (%) | | | Yield (%) | | methanol)/ |
| Catalyst | In | Co | Cu | (%) | CO | CH₄ | Methanol | Methanol | CH₄ | (mol CH₄) |
| Comparative | 2 | 3 | 0 | 16 | 21 | 4 | 75 | 12 | 0.64 | 18.75 |
| Catalyst A | | | | | | | | | | |
| InCu = 3/1 | 3 | 0 | 1 | 4.62 | 23.93 | 0.73 | 75.23 | 3.48 | 0.03 | 103.66 |
| InCoCu = 1/6/1 | 1 | 6 | 1 | 9.71 | 40.90 | 1.99 | 56.90 | 5.53 | 0.19 | 28.46 |
| InCoCu = 1/2/1 | 1 | 2 | 1 | 7.13 | 35.79 | 1.04 | 63.00 | 4.49 | 0.08 | 59.81 |
| InCoCu = 2/1/1 | 2 | 1 | 1 | 10.99 | 36.23 | 1.02 | 61.32 | 6.74 | 0.11 | 59.86 |
| InCoCu = 2/1/2 | 2 | 1 | 2 | 5.03 | 28.54 | 0.40 | 70.93 | 3.56 | 0.02 | 176.84 |
| InCoCu = 3.5/1/1 | 3.5 | 1 | 1 | 10.89 | 36.20 | 0.95 | 62.71 | 6.84 | 0.1 | 66.28 |
| InCoCu = 5/2/1 | 5 | 2 | 1 | 15.78 | 43.64 | 2.84 | 53.20 | 8.4 | 0.45 | 18.72 |
| InCoCu = 5/1/2 | 5 | 1 | 2 | 10.38 | 36.03 | 0.66 | 63.18 | 6.57 | 0.07 | 95.6 |
| InCoCu = 6/1/1 | 6 | 1 | 1 | 12.95 | 37.52 | 0.78 | 61.57 | 7.98 | 0.1 | 78.9 |
| InCoCu = 10/1/1 | 10 | 1 | 1 | 13.23 | 38.29 | 0.79 | 60.68 | 8.02 | 0.1 | 77.01 |
| InCoCu = 20/1/1 | 20 | 1 | 1 | 11.83 | 31.71 | 0.82 | 67.35 | 7.98 | 0.1 | 81.76 |
| InCoCu = 40/1/1 | 40 | 1 | 1 | 12.51 | 42.84 | 0.35 | 56.74 | 7.1 | 0.04 | 160.6 |
| InCo = 1/6 | 1 | 6 | 0 | 16.05 | 35.21 | 15.29 | 37.10 | 6.14 | 2.45 | 2.5 |
| InCo = 2/1 | 2 | 1 | 0 | 15.57 | 66.50 | 0.39 | 33.03 | 5.26 | 0.07 | 80.51 |
| InCo = 3/1 | 3 | 1 | 0 | 12.69 | 68.14 | 0.09 | 31.71 | 3.95 | 0.01 | 332.82 |
| InCo = 6/1 | 6 | 1 | 0 | 14.04 | 62.81 | 0.15 | 37.00 | 4.85 | 0.02 | 212.59 |
| InCo = 10/1 | 10 | 1 | 0 | 17.13 | 56.87 | 0.10 | 43.04 | 7.37 | 0.02 | 449.25 |

Unreduced Example Catalysts synthesized by the sol-gel method exhibit activity for CO₂ hydrogenation to methanol with significantly lower selectivity to methane compared to Comparative Catalyst A. For example, Example Catalyst InCo-10/1 and Example Catalyst InCoCu-10/1/1 yield methane selectivity of 0.10% and 0.79%, respectively, which is 40 and 5 times lower than Comparative Catalyst A. Although Comparative Catalyst A displays higher methanol selectivity compared to the unreduced Example Catalysts, Comparative Catalyst A produced significantly more methane per mole of methanol, wherein the molar ratio of methanol to CH₄ was 18.75, while Example Catalysts InCoCu-10/1/1, InCoCu-20/1/1, InCoCu-40/1/1, and InCo-10/1 exhibit molar ratios of methanol to CH₄ of 77.01, 81.76, 160.60, and 449.25, respectively.

Generally, it was observed that at the same In/Co ratio, unreduced InCo Example Catalysts exhibits higher CO₂ conversion and lower methanol selectivity than InCoCu Example Catalysts. Generally, methanol yields are higher for InCoCu Example Catalysts. Example Catalysts InCo-10/1 and InCoCu-10/1/1 are exceptions to this trend, as they have comparable methanol yields. Given these observations, results conclude that Example Catalysts InCoCu-6/1/1 and Example Catalyst InCoCu-10/1/1 exhibit the most optimal compromise between methanol selectivity, methanol yield, and molar ratio of methanol to CH₄. As evident by FIG. 1, Example Catalyst InCoCu-6/1/1 and Example Catalyst InCoCu-10/1/1 produces less methane compared to Comparative Catalyst A, with a Percent Variation increase of 311% and 321%, respectively, in the molar ratio of methanol to methane relative to Comparative Catalyst A. InCo-10/1 stands out for the impressive Percent Variation increase of 2195% in the molar ratio of methanol to methane relative to Comparative Catalyst A

### Example 2: Reduced Example Catalyst Performance

### Pretreatment

Reduced InCo Example Catalysts and Reduced InCoCu Example Catalysts were reduced under pretreatment conditions disclosed herein prior to conducting a methanol synthesis reaction in Example 2. A pretreatment was performed to reduce the catalysts before performing methanol synthesis using the reduced catalysts. The pretreatment comprised feeding a mixed gas of 50 vol. % H₂ and 50 vol. % N₂ to the reactor at a space velocity of 7.5 L_{(H2+N2)} g_{cat}⁻¹ h⁻¹L_{(H2+N2)} g_{cat}⁻¹ h⁻¹L_{(H2+N2)} g_{cathu -1} h⁻¹, the reactor at a starting reactor temperature of 100° C. Reactor temperature was increased to 350° C. for 2 hours to reduce the catalysts. Reactors were fed N₂ at a space velocity of 7500 mL_{(N2)} g_{cat}⁻¹ h⁻¹ and the reactor temperature was increased to 300° C. before performing methanol synthesis in the reactor.

### Example 2 Reactor Condition

InCo Example Catalysts and InCoCu Example Catalysts underwent pretreatment to reduce the catalysts prior to performing methanol synthesis in Example 2. After reducing the catalysts, the reactor is heated to methanol synthesis reaction conditions at 300° C. A mixed gas feed of 17.2 vol. % CO₂, 69.0 vol. % H₂, and 13.8 vol. % He, and fed to the reactor at a space velocity of 15 L_{(CO2+H2)} g_{cat}⁻¹ h⁻¹. Reactor was heated to 300° C. A membrane-based pressure controller operated with N₂ was used to pressurize the reactor to 50 bar.

### Example 2 Results

Example 2 demonstrates that Reduced InCo Example Catalysts and Reduced InCoCu Example catalysts synthesized by the sol-gel method produce less methane compared to Example Catalyst A (Table 2), which was not produced by the sol-gel method.

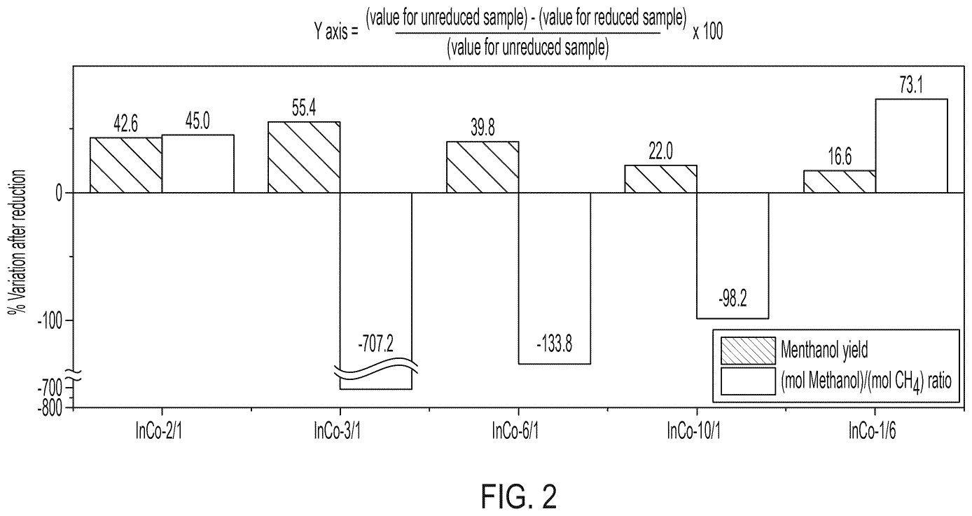

A summary of reduced catalyst performance based on measured CO₂ conversion, CO selectivity, CH₄ selectivity, CH₄OH selectivity, methanol yield, CH₄ yield, and molar ratio of methanol to CH₄ is detailed in Table 3. Tests performed under Example 2 provide catalyst performance data under identical reactor conditions to compare catalyst performance by method of synthesizing and by composition. Specifically, FIG. 2 evaluates the data from Table 3 by diagramming the percent variation in methanol yield and molar ratio of methanol to CH₄ of sol-gel synthesized catalysts, as compared to Comparative Catalyst A, which was not synthesized by sol-gel.

TABLE 3
CO₂ hydrogenation to methanol using reduced InCo Example Catalysts and
reduced InCoCu Example Catalysts, as compared to Comparative Catalyst A performance.
Conversion and selectivity obtained as the average and the error calculated as standard deviation.

| | | | | CO₂ | | | | | | (mol |
|---|---|---|---|---|---|---|---|---|---|---|
| | Molar ratio | | | Conversion | Selectivity (%) | | | Yield (%) | | methanol)/ |
| Catalyst | In | Co | Cu | (%) | CO | CH₄ | Methanol | Methanol | CH₄ | (mol CH₄) |
| InCu = 3/1 | 3 | 0 | 1 | 2.64 | 0.00 | 0.51 | 99.31 | 2.63 | 0.01 | 184.1 |
| InCoCu = 1/6/1 | 1 | 6 | 1 | 11.35 | 42.93 | 3.00 | 53.80 | 6.1 | 0.34 | 17.96 |
| InCoCu = 1/2/1 | 1 | 2 | 1 | 10.28 | 36.22 | 1.87 | 61.68 | 6.34 | 0.19 | 32.96 |
| InCoCu = 2/1/1 | 2 | 1 | 1 | 12.98 | 31.73 | 1.39 | 66.68 | 8.66 | 0.18 | 48.1 |
| InCoCu = 2/1/2 | 2 | 1 | 2 | 6.59 | 27.30 | 0.52 | 72.05 | 4.75 | 0.03 | 139.57 |
| InCoCu = 3.5/1/1 | 3.5 | 1 | 1 | 12.55 | 33.28 | 1.23 | 65.33 | 8.2 | 0.15 | 53.07 |
| InCoCu = 5/2/1 | 5 | 2 | 1 | 16.71 | 40.32 | 3.79 | 55.47 | 9.27 | 0.63 | 14.64 |
| InCoCu = 5/1/2 | 5 | 1 | 2 | 11.93 | 33.07 | 0.91 | 65.88 | 7.86 | 0.11 | 72.2 |
| InCoCu = 6/1/1 | 6 | 1 | 1 | 14.33 | 37.76 | 1.12 | 60.96 | 8.74 | 0.16 | 54.43 |
| InCoCu = 10/1/1 | 10 | 1 | 1 | 14.09 | 32.61 | 0.88 | 66.32 | 9.36 | 0.12 | 75.37 |
| InCoCu = 20/1/1 | 20 | 1 | 1 | 11.86 | 27.45 | 0.86 | 71.58 | 8.49 | 0.1 | 82.99 |
| InCoCu = 40/1/1 | 40 | 1 | 1 | 12.51 | 40.40 | 0.35 | 59.11 | 7.39 | 0.04 | 167.32 |
| InCo = 1/6 | 1 | 6 | 0 | 12.91 | 36.61 | 6.13 | 57.05 | 7.37 | 0.79 | 9.31 |
| InCo = 2/1 | 2 | 1 | 0 | 12.47 | 26.03 | 0.50 | 73.47 | 9.16 | 0.06 | 146.39 |
| InCo = 3/1 | 3 | 1 | 0 | 15.20 | 40.31 | 1.41 | 58.23 | 8.85 | 0.21 | 41.23 |
| InCo = 5/1 | 5 | 1 | 0 | 16.46 | 33.66 | 1.52 | 64.72 | 10.63 | 0.25 | 42.55 |
| InCo = 6/1 | 6 | 1 | 0 | 14.23 | 42.77 | 0.62 | 56.61 | 8.06 | 0.09 | 90.94 |
| InCo = 10/1 | 10 | 1 | 0 | 14.12 | 32.82 | 0.29 | 66.88 | 9.44 | 0.04 | 226.71 |
| InCo = 20/1 | 20 | 1 | 0 | 13.91 | 44.07 | 0.47 | 55.45 | 8.01 | 0.09 | 91.62 |

In most cases, compared to the unreduced samples with the same In/Co/Cu ratios, the reduced catalysts resulted in an increase in methanol yield and a decrease in the molar ratio of methanol to CH₄. FIG. 2 demonstrates the percent variation in methanol yield and molar ratio of methanol to CH₄ between unreduced InCo Example Catalysts and reduced InCo Example Catalysts. Percent variation after reduction may be calculated by:

$$\% Variation InCo Example Catalyst after reduction = \frac{( ( reduced InCo value ) - ( unreduced InCo value ) )}{unreduced InCo value} \times 100$$

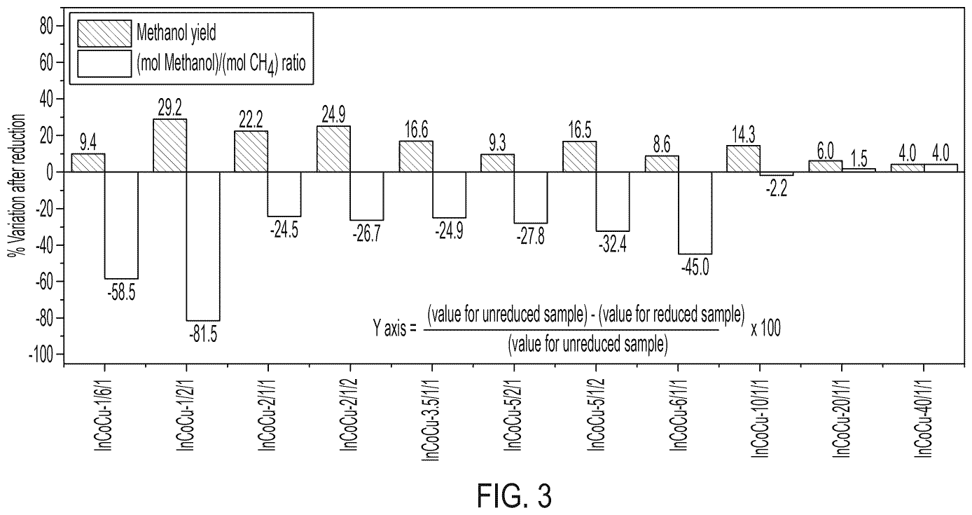

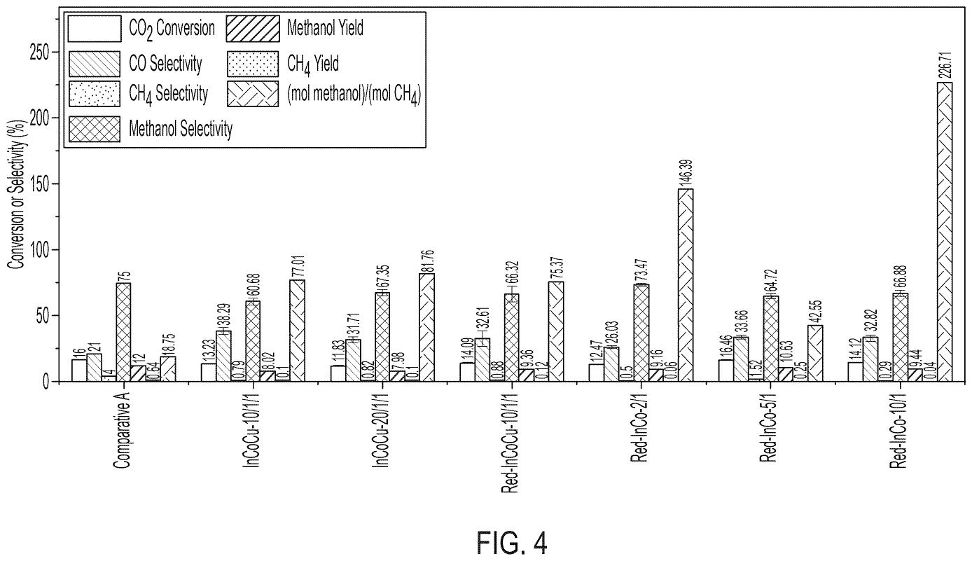

FIG. 3 demonstrates the percent variation in methanol yield and molar ratio of methanol to CH₄ between unreduced InCoCu Example Catalysts and Reduced InCoCu Example Catalysts. Percent variation after reduction may be calculated by:

$$\% Variation InCoCu Example Catalyst after reduction = \frac{( ( reduced InCoCu value ) - ( unreduced InCoCu value ) )}{unreduced InCoCu value} \times 100$$

After testing the unreduced Example Catalysts and reduced Example Catalysts disclosed hereinabove, the Example catalysts selected that displayed a methanol yield of at least 8 are: unreduced InCoCu-10/1/1, unreduced InCoCu-2/1/1, reduced InCoCu-10-1/1, reduced InCo-2/1, reduced InCo-5/1, and reduced InCo-10/1. To fairly compare catalyst performance, the molar ratio between methanol and methane was used as a normalized indicator evaluating methanol production and methane formation between catalysts. For example, while Comparative Catalyst A produces 18.75 moles of methanol per mole of methane, the sol-gel synthesized, Reduced Example Catalyst InCo-10/1 can produce 226 moles of methanol per mole of methane.

Example 2 concludes that regardless of being unreduced or reduced, both InCo Example Catalysts and InCoCu Example Catalysts synthesized by the sol-gel method disclosed herein outperform Comparative Catalyst A, which was synthesized without the sol-gel method.

### Example 3: Reduced Catalyst Performance with Contamination

Example 3 demonstrates that Example Catalysts synthesized by the sol-gel method are compatible to the presence of common contaminants. Further, Example 3 demonstrates that, even when exposed to common contaminants, reduced InCo Example Catalysts, reduced InCoCu Example catalysts, unreduced InCo Example Catalysts, and unreduced InCoCu Example catalysts synthesized by the sol-gel method produce less methane compared to Example Catalyst A, which was not synthesized by the sol-gel method.

InCo Example Catalysts and InCoCu Example Catalysts underwent pretreatment to reduce the catalysts prior to performing methanol synthesis Example 3. Reduced InCo Example Catalysts and Reduced InCoCu Example Catalysts were used in Example 3.

Reduced InCo Example Catalysts and Reduced InCoCu Example Catalysts were tested for performance by applying the catalysts to a methanol synthesis reaction, wherein the conditions of each methanol synthesis reaction performed comprises a mixed gas feed, specific contaminant conditions, and specific L_{(CO2+H2)} g_{cat}⁻¹ h⁻¹ flow. Each contamination reaction performed was pressurized with a membrane-based pressure controller operated with N₂, used to pressurize the reactor to 50 bar.

Typical contaminants in a methanol synthesis reaction may comprise oxygen, toluene, methane, and carbon monoxide. Example 3 was performed on catalysts to measure performance of catalysts when introduced to contaminants. To simulate contaminants in an experimental environment, contaminants were co-fed with the mixed gas feed. Contaminants were tested separately, in which catalyst performance data was collected separately for each contaminant. The contaminants tested were: oxygen, methane, toluene, and carbon monoxide.

### Example 3 Reactor Conditions

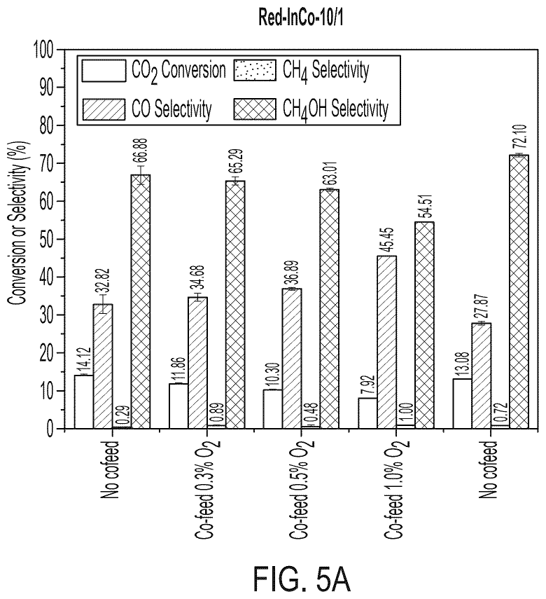

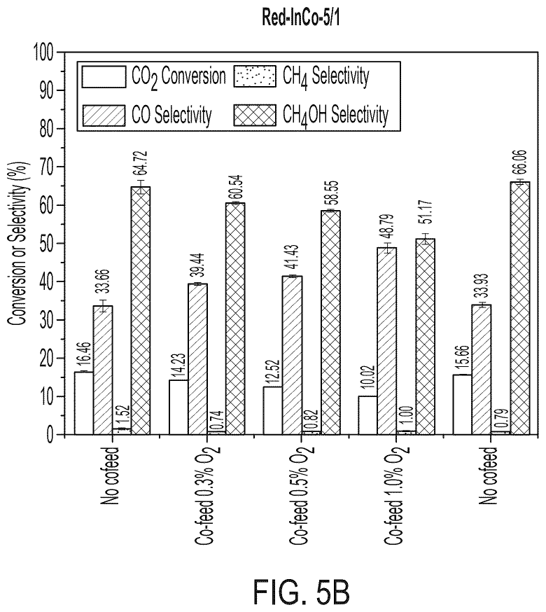

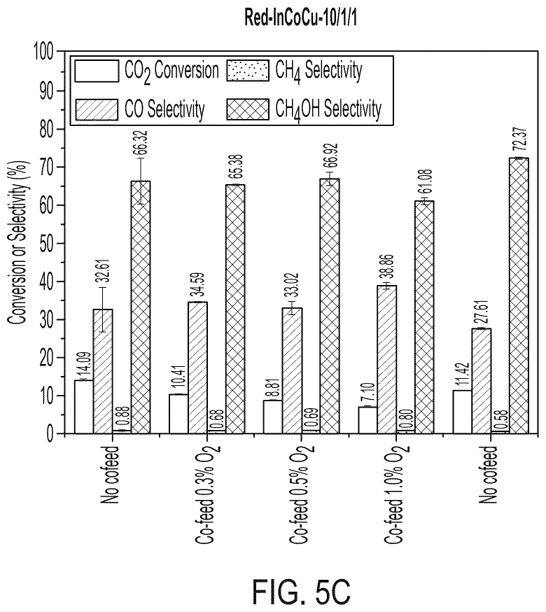

For the oxygen contaminant tests in particular, air was co-fed to the mixed gas feed, mixed gas feed fed at a GHSV of 15 L_{(CO2+H2)} g_{cat}⁻¹ h⁻¹, with airflow adjusted to provide oxygen concentration as needed. Airflow was adjusted to achieve an oxygen concentration of 0.3 vol. % to measure CO₂ conversion, CO selectivity, CH₄ selectivity, and CH₄OH selectivity of reduced InCo-10/1, reduced InCo-5/1, and reduced InCoCu-10/1/1 at 0.3 vol. % oxygen contamination. In a separate test, airflow was adjusted to achieve an oxygen concentration of 0.5 vol. % to measure CO₂ conversion, CO selectivity, CH₄ selectivity, and CH₄OH selectivity of reduced InCo-10/1, reduced InCo-5/1, and reduced InCoCu-10/1/1 at 0.5 vol. % oxygen contamination. In a separate test, airflow was adjusted to achieve an oxygen concentration of 1.0 vol. % to measure CO₂ conversion, CO selectivity, CH₄ selectivity, and CH₄OH selectivity of reduced InCo-10/1 (FIG. 5A), reduced InCo-5/1 (FIG. 5B), and reduced InCoCu-10/1/1 (FIG. 5C) at 1.0 vol. % oxygen contamination. FIG. 5A, FIG. 5B, and FIG. 5C summarize the results of the three oxygen concentration tests with each respective catalyst.

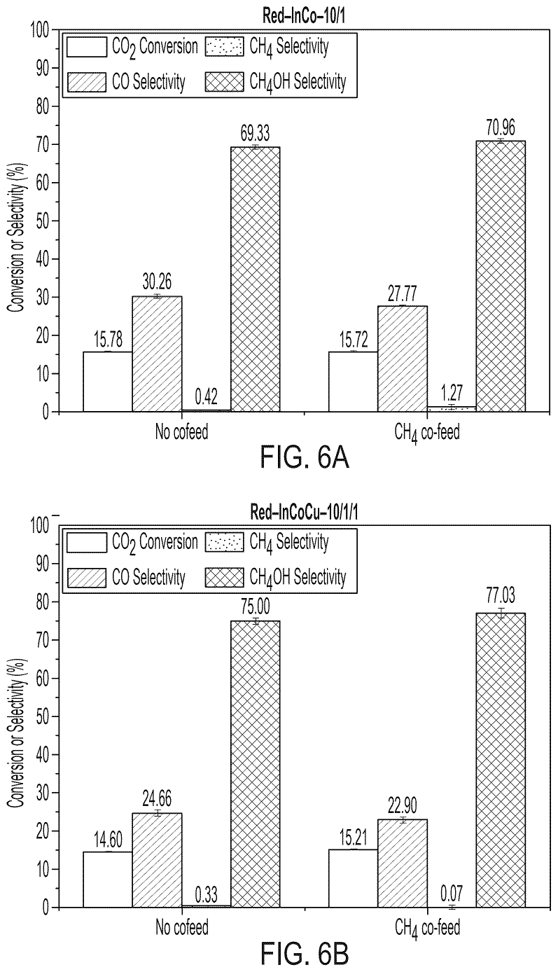

For the methane contaminant tests in particular, a pure flow stream of methane was co-fed to the mixed gas feed, mixed gas feed fed at a GHSV of 7.5 L_{(CO2+H2)} g_{cat}⁻¹ h⁻¹. Specifically, to reach CO₂/CH₄ ratio of 9:1, pure methane was added such that the GHSV of pure methane to mixed gas feed was approximately 0.17 L_{(CH4)} g_{cat}⁻¹ h⁻¹. Reactions measure CO₂ conversion, CO selectivity, CH₄ selectivity, and CH₄OH selectivity of reduced InCo-10/1 and reduced InCoCu-10/1/1 at CO₂/CH₄ ratio of 9:1. A separate comparison test is run without co-feeding methane to directly compare reduced InCo-10/1 performance and reduced InCoCu-10/1/1 performance with and without contamination. FIG. 6A and FIG. 6B summarize the results of the methane contaminant tests with reduced InCo-10/1 and reduced InCo-10/1/1, respectively.

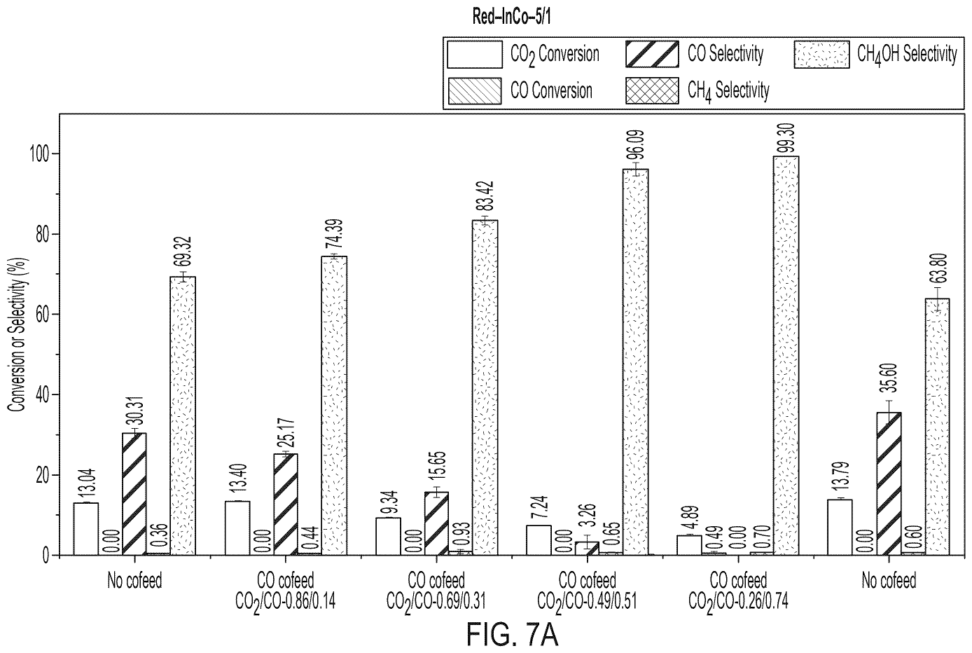

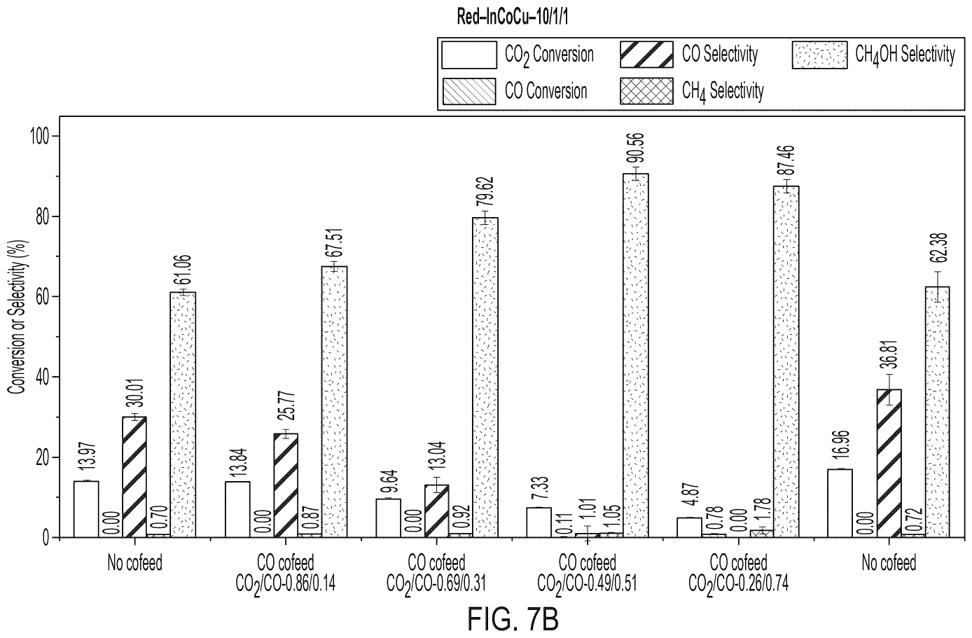

For the carbon monoxide contaminant test in particular, a contaminant feed mixture comprising H₂, CO₂, and CO was co-fed to the mixed gas feed, the mixed gas feed at a GHSV of 15 L_{(CO2+H2)} g_{cat}⁻¹ h⁻¹. The feed was composed by a mixture of H₂/(CO₂+CO) in a volumetric ratio of 4/1. The concentration of carbon dioxide and carbon monoxide in the reaction was adjusted by altering the CO₂/CO ratio. Four carbon monoxide concentrations were tested at corresponding ratios of CO₂/CO at 0.86/0.14, 0.65/0.31, 0.49/0.51, and 0.26/0.79. FIG. 7A and FIG. 7B summarizes the results of the carbon monoxide contaminant tests with reduced InCo-5/1 and reduced InCoCu-10/1/1, respectively.

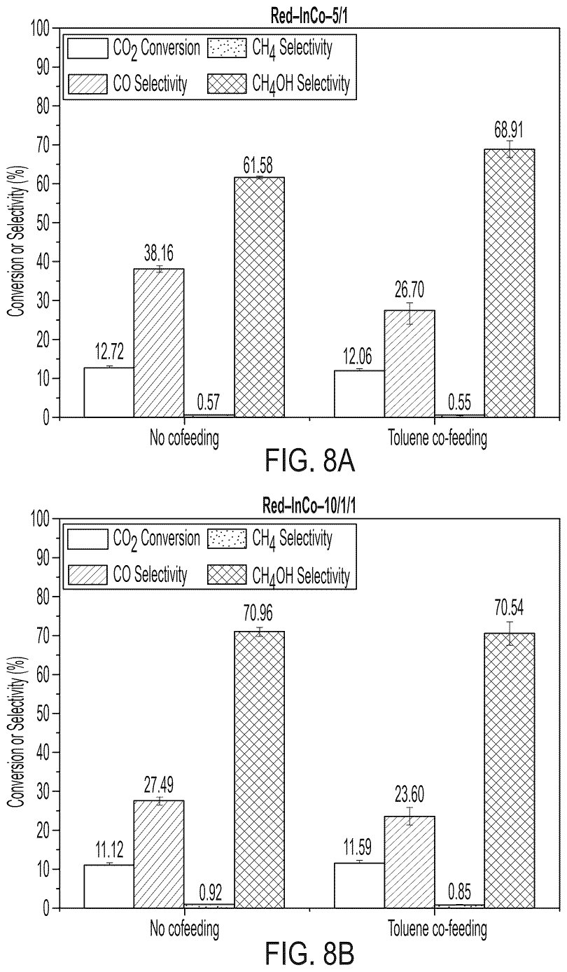

For the toluene contaminant tests in particular, a pure flow stream of toluene was co-fed to the mixed gas feed, mixed gas feed fed at a GHSV of 22 L_{(CO2+H2)} g_{cat}⁻¹ h⁻¹. Specifically, pure toluene was added to reach a toluene concentration of 0.2 vol. % based on the total flow. Reactions measure CO₂ conversion, CO selectivity, CH₄ selectivity, and CH₄OH selectivity of reduced InCo-5/1 and reduced InCoCu-10/1/1 at 0.2 vol. % toluene contamination. A separate comparison test is run without co-feeding toluene to directly compare reduced InCo-5/1 performance and reduced InCoCu-10/1/1 performance with and without contamination. FIG. 8A and FIG. 8B summarizes the results of the toluene contaminant tests with reduced InCo-5/1 and reduced InCoCu-10/1/1, respectively.

### Example 3 Results

Results of Example 3 demonstrate that Example Catalysts synthesized by the sol-gel method are compatible to the presence of common contaminants, the contaminants comprising: oxygen, methane, carbon monoxide, and toluene.

An increase in oxygen partial pressure resulted in a reduction of carbon dioxide conversion, as well as an increase in carbon monoxide selectivity, at the expense of methanol selectivity, as observed in FIG. 5A, FIG. 5B, and FIG. 5C. Importantly, CH₄ selectivity was minimally affected. Example Catalysts fully regained their initial catalytic performance after exposure to oxygen contamination, which confirmed the stability of the catalyst in the presence of oxygen contaminant

Contaminants comprising methane had little effect on catalytic performance of the sol-gel synthesized Example Catalysts, as seen in FIG. 6A and FIG. 6B.

An increase in carbon monoxide concentration resulted in a reduction of carbon dioxide conversion, as observed in FIG. 7. Carbon monoxide also hindered the reverse water gas shift reaction, which reduced the selectivity to carbon monoxide. Significant CO conversion was only observed when feeding the reactor with a ratio of carbon dioxide to carbon monoxide of 0.26:0.74. Importantly, carbon monoxide resulted in a slight increase of CH₄ selectivity, but a CH₄ selectivity below 1% was maintained through all carbon monoxide concentrations tested.

Contaminants comprising toluene did not affect the performance of Example Catalysts synthesized by the sol-gel method, as evident in FIG. 8A and FIG. 8B. However, after removing contact between the Example Catalyst and the contaminant comprising toluene, a slight increase in methanol selectivity was observed for reduced Example Catalyst InCo-5/1.

In commercial applications CO, CH₄, aromatics, and O₂ can be present in the feedstock stream. InCo Example Catalysts and InCoCu Example Catalysts demonstrate compatibility with these contaminants. Furthermore, little effect has been observed on methane selectivity, confirming that reduced InCo Example Catalysts and InCoCu Example Catalysts display low selectivity to methane even in the presence of contaminants.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for preparing catalyst particles comprising:
preparing a precursor solution comprising a solvent, an indium-based composition, and at least one of a cobalt-based composition and a copper-based composition;
contacting the precursor solution with an acidic solution to form a sol;
heating the sol to form a gel;
drying the gel to yield a solid material; and
calcining the solid material to form catalyst particles, wherein the catalyst particles comprise a composite having at least one of an indium oxide, an indium hydroxide, or combinations thereof, and at least one of a copper oxide, a copper hydroxide, a cobalt oxide, a cobalt hydroxide, or combinations thereof.

2. The method of claim 1, wherein the indium-based composition, the cobalt-based composition, the copper-based composition, or all three, comprise water-soluble salts.

3. The method of claim 1, wherein the acidic solution comprises an organic acid.

4. The method of claim 3, wherein the organic acid comprises citric acid.

5. The method of claim 1, wherein the precursor solution comprises from 12.5 mol. % to 95.2 mol. % indium, based on a total metal content.

6. The method of claim 1, wherein the precursor solution comprises up to 85.7 mol. % cobalt, based on a total metal content.

7. The method of claim 1, wherein the precursor solution comprises up to 40 mol. % cobalt, based on a total metal content.

8. The method of claim 1, wherein the sol is heated at from 50° C. to 90° C. to form the gel.

9. The method of claim 1, wherein the gel is dried at from 90° C. to 150° C. under static conditions to form a solid.

10. The method of claim 1, wherein the calcining takes place at from 250° C. to 500° C.

11. The method of claim 1, the method further comprising a reduction step, the reduction step comprising:
contacting the catalyst particles to a reduction gas feed under an initial temperature and increasing to a final temperature to form reduced catalyst particles, wherein:
the reduction gas feed comprises up to 100 vol. % hydrogen;
the initial reduction temperature is from 25° C. to 175° C.; and
the final reduction temperature is from 250° C. to 600° C.

12. The method of claim 11, wherein the reduction gas feed further comprises from 25 vol. % to 95 vol. % nitrogen.

13. The method of claim 11, wherein the reduction step duration is from 0.5 to 10 hours.

14. The method of claim 11, wherein the reduction gas feed enters the reactor at a space velocity of from 5 to 100 L_{(H2+N2)} g_{cathu -1} h⁻¹.

15. A process for producing methanol by hydrogenation with the catalyst particles formed by the method of claim 1, the process comprising:
contacting a mixed gas feed to the catalyst particles at a reaction temperature, the mixed gas feed comprising carbon dioxide and hydrogen to form a product stream comprising methanol.

16. A process for producing methanol by hydrogenation with the catalyst particles formed by the method of claim 11, the process comprising:
contacting a mixed gas feed to the reduced catalyst particles at a reaction temperature, the mixed gas feed comprising carbon dioxide and hydrogen, to form a product stream comprising methanol.

17. A process for producing methanol by hydrogenation with the catalyst particles formed by the method of claim 1, the process comprising:
contacting a mixed gas feed to the catalyst particles at a reaction temperature, the mixed gas feed comprising carbon dioxide and hydrogen, to form a product stream comprising methanol and methane,
wherein the selectivity of methane is less than 3.5%, the molar ratio of methanol to methane is from 2:1 to 450:1, or both.

18. A process for producing methanol by hydrogenation with the catalyst particles formed by the method of claim 11, the process comprising:
contacting a mixed gas feed to the catalyst particles at a reaction temperature, the mixed gas feed comprising carbon dioxide and hydrogen, to form a product stream comprising methanol and methane,
wherein the selectivity of methane is less than 3.5%, the molar ratio of methanol to methane is from 2:1 to 450:1, or both.

19. The process of claim 17, wherein the selectivity of methane is less than 1%.

20. The process of claim 18, wherein the selectivity of methane is less than 1%.
